(19) United States
(12) Patent Application Publication
(10) Pub. No.: US 2004/0120977 A1
(43) Pub. Date: Jun. 24, 2004

(54) Anti-inflammatory coatings for implantable medical devices and devices containing said coatings

(76) Inventor: John H. Dodd, Stockton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/732,570

(22) Filed: Dec. 10, 2003

Related U.S. Application Data
(60) Provisional application No. 60/434,007, filed on Dec. 17, 2002.
(60) Provisional application No. 60/482,273, filed on Jun. 25, 2003.

(51) Int. Cl. A61F 2/06; A61K 31/4747; A61K 31/4164

(52) U.S. Cl. 424/423; 623/1.46; 514/389; 514/424; 514/278; 514/409

(57) ABSTRACT

The present invention relates to implantable surgical medical devices having coatings comprising one or more compounds that inhibit TNF-α converting enzyme (TACE), more particularly, stents having coatings comprising TACE inhibitors.

### CROSS-REFERENCE TO RELATED APPLICATIONS

The present application claims the priority benefits of U.S. Provisional Application No. 60/434,007, filed Dec. 17, 2002, and U.S. Provisional Application No. 60/482,273, filed Jun. 25, 2003, which are expressly incorporated fully herein by reference.

### FIELD OF THE INVENTION

This invention relates to implantable surgical medical devices having coatings comprising one or more compounds that inhibit TNF-α converting enzyme (TACE), more particularly, stents having coatings comprising TACE inhibitors.

### BACKGROUND OF THE INVENTION

Implantable medical devices are used in various medical procedures. Such devices include, without limitation, stents, catheters, sutures, meshes, vascular grafts, shunts, and filters for removing emboli.

The use of stents in medical procedures is rapidly increasing. Stents are now commonly used in translumenial procedures such as angioplasty to restore adequate blood flow to the heart and other organs. Stents may be used to prop open arteries in connection with heart surgery, to aid in drug delivery, and to provide natural routes for access, without open surgery, in performing vascular reconstructions. Recently, there have been improvements in stent designs and implantation techniques, leading to broader applications for their use and a reduction of complications.

Generally, stents are cylindrical devices perforated with passages. The stents may be composed of helically wound or serpentine wire structures in which the spaces between the wires form passages. Or they may be flat perforated structures that are subsequently rolled to form tubular or cylindrical structures that are woven, wrapped, drilled, etched, or cut to form passages. Examples of stents may be found, for example in U.S. Pats. Nos. 4,733,665; 4,800,882; 4,886,062; 5,514,154; and 6,190,403, the entire contents of which are incorporated herein by reference.

Despite the advantages and increasing importance of stents, their introduction into the body may stimulate foreign body reactions that result in thrombosis or restinosis. Additionally, the replacement of stents (e.g., bilary stents, arterial stents), or other devices (e.g., mitral valves, joint replacements, and pacemakers), can cause localized acute and chromic inflammation which stimulates cell recruitment and tissue proliferation. Restinosis can occur following the placement of a bare metal arterial stent and can be a serious consequence in up to 50% of patients receiving them.

Various techniques have been attempted to reduce the high restinosis rates associated with stents and medical devices. For example, radioactive isotopes have been incorporated into metal stents, and a variety of coatings and compositions have been proposed to reduce complications. In some instances, the coatings are designed to reduce the stimulus that the stent provides to the injured lumen wall, or alternatively, they may be designed to deliver a pharmaceutical or therapeutic agent to the lumen that reduces smooth muscle tissue proliferation or restinosis. The mechanism for delivery of the pharmaceutical agent may be via diffusion of the agent through a bulk polymer coating the stent, through pores that are created in the polymer structure, or by erosion of a biodegradable coating.

For example, a bioabsorbable or biodegradable material may be applied as a coating on a stent, with the bioaborbable or biodegradable material either encapsulating the pharmaceutical agent, or binding the agent to the stent surface. In these cases, once the stent is placed in contact with the body, the bioabsorbable or biodegradable material will become absorbed or degrade, thereby releasing the pharmaceutical agent. Additionally, a top coating may be applied to delay the release of the pharmaceutical agent, or can be used as a matrix for delivery of the agent. Layering of coatings may be used to stagger the release of the pharmaceutical agent or to control the release of different agents placed in different layers. Suitable materials for use in making these coatings as well as methods for coating the stents are described in U.S. Pat. Nos. 5,356,433, 5,213,898, 5,049,403, 4,807,784 and 4,565,740, each of which is incorporated herein by reference.

There is experimental evidence that the degree of inflammation and neointimal wall formation is proportional to the degree of penetration which occurs from the stent struts protuding into the vessel wall. The major cytokines assumed to be released during the acute inflammation which occurs after stent placement is IL-1, IL-6 and TNF-alpha. In addition to acute responses, chronic inflammation also plays a major role in the stimulation of tissue proliferation. Accumulated macrophages continually release TNF-alpha which in addition to direct inflammatory effects, synergizes with IFN-gamma resulting in a greater effect than can be found when either agent acts alone. Considering the major role that TNF-alpha plays in acute inflammation and the pivotal role it plays both directly and in synergy with WFN-gamma in chronic inflammation, agents which block TNF-alpha production and/or release could be useful in inhibiting undesirable inflammatory and proliferative responses associated with non biological implants. These agents would need to be included in a drug eluting matrix surrounding the device or coated on some way directly on the implant in such a way that the release rate would allow maximal efficacy.

It would be advantageous to provide coatings for implantable medical devices that will reduce thrombosis, restinosis, and other adverse reactions. The present invention provides anti-inflammatory coatings for such medical devices that include use of an effective amount of a TACE inhibitor or its combination with one or more additional therapeutic agents.

### SUMMARY OF THE INVENTION

According to one aspect of the invention, there is provided a medical device for implanting into a mammalian body wherein the medical device has a coating material comprising an amount of a TACE inhibitor effective for reducing restinosis, or a pharmaceutically-acceptable salt, hydrate, or prodrug of the TACE inhibitor. Preferably, the implantable medical device is a stent. According to another aspect of the invention, the coating for the stent or other implantable medical device comprises a TACE inhibitor having the formula selected from (I), (II), (III), (IV), (V), and (VI) (shown below), or a pharmaceutically-acceptable salt, hydrate, or prodrug thereof.

### DETAILED DESCRIPTION OF THE INVENTION

Applicant has surprisingly discovered that use of a TACE inhibitor for making a coating on an implantable medical device, more particularly, on a stent, is particularly advantageous in reducing the restinosis or thrombosis associated with introduction of the stent into the mammalian body.

Besides the TACE inhibitor, one or more additional therapeutic agents may be incorporated into the stent coating to provide an additive or synergistic therapeutic advantage. For example, such additional therapeutic agents include, but not limited to: antiproliferative/antimitotic agents including natural 12 products such as vinca alkaloids (i.e., vinblastine, vincristine, and vinorelbine), paclitaxel, epidipodophyllotoxins (i.e., etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L asparagine and deprives cells which don't have the capacity to synthesize their own asparagine); antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and is analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2 chlorodeoxyadenosine(cladribine)); platinum coordination complexes (cisplatin, carboplatin), procarbazine, hydroxyurea, mitotane, aminoglutethimide; hormones (i.e.estrogen); Anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); antiinflammatory: such as adrenocortical steroids (cortisol, cortisone, fludrocortisone, prednisone, prednisclone, 6(x methylprednisolone, triamcinolone, betamethasone, and dexamethasone), non-steroidal agents (salicylic acid 13 derivatives i.e., aspirin; para-aminophenol derivatives i.e. acetomninophen; Indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); Angiogenic agents: vascular endothelial growth factor (VEGF), fibroblast growth factor (FGF); nitric oxide donors; cell cycle inhibitors; mTOR inhibitors; growth factor signal transduction knase inhibitors; anti sense oligonucleotide; prodrug molecules; and combinations thereof.

Said TACE inhibitors useful in the present invention are compounds of Formula (I), (II), (III), (IV), (V) or (VI):

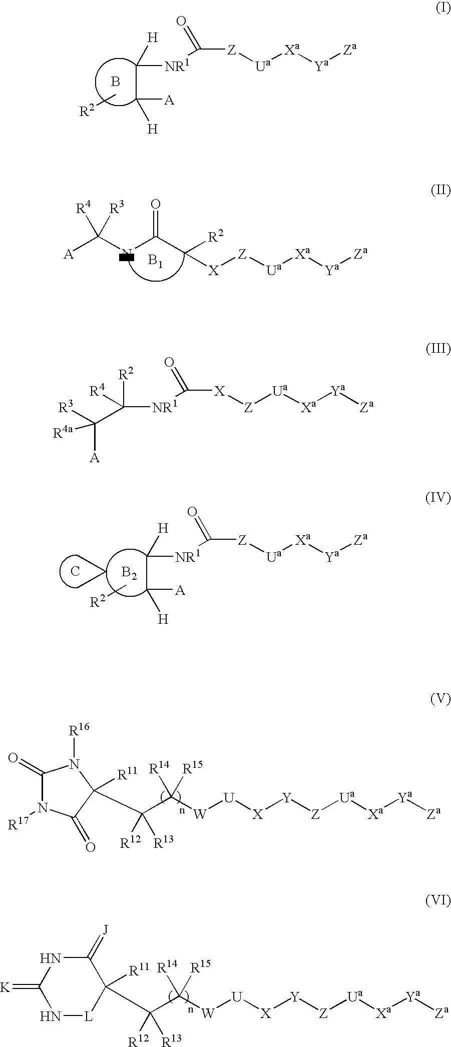

or a stereoisomer or a pharmaceutically acceptable salt, hydrate, or prodrug thereof, wherein;

A, at each occurrence, is independently selected from —COR⁵, —CO₂H, —CH₂CO₂H, —CONHOH, —CONHOR⁵, —CONHOR⁶, —N(OH)COR⁵, —SH, and —CH₂SH;

ring B is a 4-7 membered non-aromatic ring comprising: carbon atoms, 0-1 carbonyl group, 0-1 double bond, and 0-2 ring heteroatoms selected from O, N, NR¹⁰ and substituted with 0-3 R^{c}; provided that ring B contains other than a O—O or N—O bond;

ring B₁ is a 4-7 membered cyclic amide comprising: carbon atoms, 0-2 additional heteroatoms selected from O, NR¹⁰, and S(O)ₚ, 0-1 additional carbonyl group, and 0-1 double bond;

ring B₂ is a 4-7 membered non-aromatic carbocycle or heterocycle comprising: carbon atoms, 0-1 carbonyl group, 0-1 double bond, and 0-2 ring heteroatoms selected from O, N, and NR¹⁰, provided that ring B contains other than a O—O bond;

ring C forms a spiro ring on Ring B₂ and is a 4-10 membered carbocycle substituted with 0-3 R^{g} or a 4-10 membered heterocycle comprising: carbon atoms, 0-3 carbonyl groups, 0-4 double bonds, and 0-4 ring heteroatoms selected from O, N, NR¹⁰, and S(O)ₚ and substituted with 0-3 R^{g}, provided that ring C contains other than a S—S, O—O, or S—O bond;

J is O or S;

K is O or S;

L is C(═O), C(═S) or CH₂;

U, at each occurrence, is absent or is independently selected from O, NR^{a}, C(O), C(O)O, C(O)NR^{a}, NR^{a}C(O), S(O)ₚ, and S(O)ₚNR^{a};

X, at each occurrence, is absent or is independently C₁₋₄ alkylene, C₂₋₄ alkenylene, or C₂₋₄ alkynylene;

Y, at each occurrence, is absent or is independently O, NR^{a}, S(O)ₚ, or

W is (CR^{a}R^{a1})ₘ, C₂₋₃ alkenylene, or C₂₋₃ alkynylene;

Z, at each occurrence, is independently selected from a C₃₋₆ carbocycle substituted with 0-4 R^{b} and a 5-6 membered heterocycle comprising: carbon atoms, 1-4 heteroatoms selected from the group consisting of N, O, and S(O)ₚ, and substituted with 0-5 R^{b};

U^{a}, at each occurrence, is absent or is independently selected from: O, NR^{a}, C(O), C(O)O, C(O)NR^{a}, NR^{a}C(O), S(O)ₚ, and S(O)ₚNR^{a};

X^{a}, at each occurrence, is absent or is independently C₁₋₄ alkylene, C₂₋₄ alkenylene, or C₂₋₄ alkynylene;

Y^{a}, at each occurrence, is absent or is independently O or NR^{a};

Z^{a}, at each occurrence, is independently selected from H, a C₃₋₁₀ carbocycle substituted with 0-5 R^{c} and a 5-10 membered heterocycle comprising: carbon atoms, 1-4 heteroatoms selected from the group comprising N, O, and SOₚ, and substituted with 0-5 R^{c};

provided that Z, U^{a}, Y^{a}, and Z^{a} do not combine to form a N—N, N—O, O—N, O—O, S(O)ₚ—O, O—S(O)ₚ or S(O)ₚ—S(O)ₚ group;

R¹, at each occurrence, is independently selected from H, C₁₋₄ alkyl, phenyl and benzyl;

R², at each occurrence, is independently selected from Q, C₁₋₆ alkylene-Q, C₂₋₆ alkenylene-Q, C₂₋₆ alkynylene-Q, —(CR^{a}R^{a1})ᵣ₁O(CR^{a}R^{a1})ᵣ-Q, —(CR^{a}R^{a1})ᵣ ₁NR^{a}(CR^{a}R^{a1})ᵣ-Q, —(CR^{a}R^{a1})ᵣ₁C(O)(CR^{a}R^{a1})ᵣ-Q, —(CR^{a}R^{a1})ᵣ ₁C(O)O(CR^{a}R^{a1})ᵣ-Q, —(CR^{a}R^{a1})ᵣC(O)NR^{a}R^{a1}, —(CR^{a}R^{a1})ᵣ ₁C(O)NR^{a}(CR^{a}R^{a1})ᵣ-Q, —(CR^{a}R^{a1})ᵣ S(O)ₚ(CR^{a}R^{a1})ᵣ-Q, and —(CR^{a}R^{a1})ᵣ₁SO₂NR^{a}(CR^{a}R^{a1})ᵣ-Q;

R³, at each occurrence, is independently selected from H, C₁₋₆ alkylene-Q, C₂₋₆ alkenylene-Q, C₂₋₆ alkynylene-Q, —(CH₂)ᵣ₁O(CH₂)ᵣ-Q, —(CH₂)ᵣ₁NR^{a}(CH₂)ᵣ-Q, —(CH₂)ᵣ₁C(O)(CH₂)ᵣ-Q, —(CH₂)ᵣ₁C(O)NR^{a}(CH₂)ᵣ-Q, —(CH₂)ᵣ₁NR^{a}C(O)(CH₂)ᵣ-Q, —(CH₂)ᵣ₁C(O)NR^{a}(CH₂)ᵣ-Q, —(CH₂)ᵣ₁NR^{a}C(O)O(CH₂)ᵣ-Q, —(CH₂)ᵣ₁NR^{a}C(O)NR^{a}(CH₂)ᵣ-Q, —(CH₂)ᵣ₁S(O)ₚ(CH₂)ᵣ-Q, —(CH₂)ᵣ₁SO₂NR^{a}(CH₂)ᵣ-Q, —(CH₂)ᵣ₁NR^{a}SO₂(CH₂)ᵣ-Q, and —(CH₂)ᵣ₁NR^{a}SO₂NR^{a}(CH₂)ᵣ-Q;

Q, at each occurrence, is independently selected from H, a C₃₋₆ carbocycle substituted with 0-5 R^{d} and a 5-10 membered heterocycle comprising: carbon atoms and 1-4 heteroatoms selected from the group consisting of N, O, and S(O)ₚ, and substituted with 0-5 R^{d};

R⁴, at each occurrence, is independently H or C₁₋₆ alkyl;

R^{4a} is H or C₁₋₆ alkyl;

alternatively, R³ and R⁴ in Formula (II), together with the carbon atom to which they are attached, combine to form a 3-6 membered carbocycle or heterocycle comprising: carbon atoms and 0-2 ring heteroatoms selected from O, N, NR¹⁰, and S(O)ₚ, and substituted with 0-1 R^{c};

alternatively, R¹ and R² in Formula (III), together with the carbon and nitrogen atoms to which they are attached, combine to form a 3-10 membered heterocycle comprising: carbon atoms and, in addition to the nitrogen atom to which R¹ is attached, 0-1 ring heteroatoms selected from O, N, NR¹⁰, and S(O)ₚ, and substituted with 0-1 R^{c};

alternatively, R¹ and R³ in Formula (III), together with the carbon and nitrogen atoms to which they are attached, combine to form a 4-6 membered heterocycle comprising: carbon atoms and, in addition to the nitrogen atom to which R¹ is attached, 0-1 ring heteroatoms selected from O, N, and NR¹⁰, and substituted with 0-1 R^{c};

alternatively, R² and R⁴ in Formula (III), together with the carbon atom to which they are attached, combine to form a 3-10 membered carbocycle or heterocycle comprising: carbon atoms and 0-2 ring heteroatoms selected from O, N, NR¹⁰, and S(O)ₚ, and substituted with 0-3 R^{c};

alternatively, R³ and R^{4a} in Formula (III), together with the carbon atom to which they are attached, combine to form a 3-6 membered carbocycle or heterocycle comprising: carbon atoms and 0-2 ring heteroatoms selected from O, N, NR¹⁰, and S(O)ₚ and substituted with 0-1 R^{c};

R⁵, at each occurrence, is independently selected from C₁₋₆ alkyl substituted with 0-2 R^{b}, and C₁₋₄ alkyl substituted with 0-2 R^{e};

R⁶, at each occurrence, is independently selected from phenyl, naphthyl, C₁₋₁₀ alkyl-phenyl-C₁₋₆ alkyl-, C₃₋₁₁ cycloalkyl, C₁₋₆ alkylcarbonyloxy-C₁₋₃ alkyl-, C₁₋₆ alkoxycarbonyloxy-C₁₋₃ alkyl-, C₂₋₁₀ alkoxycarbonyl, C₃₋₆ cycloalkylcarbonyloxy-C₁₋₃ alkyl-, C₃₋₆ cycloalkoxycarbonyloxy-C₁₋₃ alkyl-, C₃₋₆ cycloalkoxycarbonyl, phenoxycarbonyl, phenyloxycarbonyloxy-C₁₋₃ alkyl-, phenylcarbonyloxy-C₁₋₃ alkyl-, C₁₋₆ alkoxy-C₁₋₆ alkylcarbonyloxy-C₁₋₃ alkyl-, [5-(C₁-C₅ alkyl)-1,3-dioxa-cyclopenten-2-one-yl]methyl, [5-(R^{a})-1,3-dioxa-cyclopenten-2-one-yl]methyl, (5-aryl-1,3-dioxa-cyclopenten-2-one-yl)methyl, —C₁₋₁₀ alkyl-NR⁷R^{7a}, —CH(R⁸)OC(═O)R⁹, and —CH(R⁸)OC(═O)OR⁹;

R⁷, at each occurrence, is independently H, C₁₋₆ alkyl, C₂₋₆ alkenyl, C₃₋₆ cycloalkyl-C₁₋₃ alkyl-, or phenyl-C₁₋₆ alkyl-;

R^{7a}, at each occurrence, is independently H, C₁₋₆ alkyl, C₂₋₆ alkenyl, C₃₋₆ cycloalkyl-C₁₋₃ alkyl-, or phenyl-C₁₋₆ alkyl-;

R⁸, at each occurrence, is independently H or C₁₋₄ linear alkyl;

R⁹, at each occurrence, is independently selected from H, C₁₋₆ alkyl substituted with 1-2 R^{f}, C₃₋₆ cycloalkyl substituted with 1-2 R^{f}, and phenyl substituted with 0-2 R^{b};

R¹⁰, at each occurrence, is independently selected from H, —(CR^{a}R^{a1})ₜNR^{a}R^{a1}, —(CR^{a}R^{a1})ᵣ₁C(O)NR^{a}OH, —(CR^{a}R^{a1})ᵣ₁C(O)(CR^{a}R^{a1})ᵣR^{h}, —(CR^{a}R^{a1})ᵣC(O)OR^{a1}, —(CR^{a}R^{a1})ᵣC(S)OR^{a1}, —(CR^{a}R^{a1})ᵣC(O)NR^{a}R^{a1}, —(CR^{a}R^{a1})ₜNR^{a}C(O)R^{a1}, —(CR^{a}R^{a1})ᵣC(S)NR^{a}R^{a1}, —(CR^{a}R^{a1})ₜOC(O)NR^{a}R^{a1}, —(CR^{a}R^{a1})ₜNR^{a}C(O)OR^{a1}, —(CR^{a}R^{a1})ₜNR^{a}C(O)NR^{a}R^{a1}, —(CR^{a}R^{a1})ᵣS(O)ₚR^{a2}, —(CR^{a}R^{a1})ᵣSO₂NR^{a}R^{a1}, —(CR^{a}R^{a1})ₜNR^{a}SO₂R^{a2}, —(CR^{a}R^{a1})ₜNR^{a}SO₂NR^{a}R^{a1}, C₁₋₆ alkyl substituted with 0-2 R^{c1}, C₂₋₆ alkenyl substituted with 0-2 R^{c1}, C₂₋₆ alkynyl substituted with 0-2 R^{c1}, —(CR^{a}R^{a1})ᵣ—C₃₋₁₀ carbocycle substituted with 0-2 R^{c1}, and —(CR^{a}R^{a1})ᵣ-5-10 membered heterocycle consisting of carbon atoms and 1-4 heteroatoms selected from the group consisting of N, O, and S(O)ₚ, and substituted with 0-2 RCI;

R¹¹ is independently selected from Q, C₁₋₆ alkylene-Q, C₂₋₆ alkenylene-Q, C₂₋₆ alkynylene-Q, —(CR^{a}R^{a1})ᵣ₁O(CR^{a}R^{a1})ᵣ-Q, —(CR^{a}R^{a1})ᵣ₁NR^{a}(CR^{a}R^{a1})ᵣ-Q, —(CR^{a}R^{a1})ᵣ₁C(O)(CR^{a}R^{a1})ᵣ-Q, —(CR^{a}R^{a1})ᵣ₁C(O)O(CR^{a}R^{a1})ᵣ-Q, —(CR^{a}R^{a1})ᵣC(O)NR^{a}R^{a1}, —(CR^{a}R^{a1})ᵣ₁C(O)NR^{a}(CR^{a}R^{a1})ᵣ-Q, —(CR^{a}R^{a1})ᵣ S(O)ₚ(CR^{a}R^{a1})ᵣ-Q, and —(CR^{a}R^{a1})ᵣ₁SO₂NR^{a}(CR^{a}R^{a1})ᵣ-Q;

R¹², at each occurrence, is independently selected from Q, C₁₋₆ alkylene-Q, C₂₋₆ alkenylene-Q, C₂₋₆ alkynylene-Q, —(CR^{a}R^{a1})ᵣ₁O(CR^{a}R^{a1})ᵣ-Q, —(CR^{a}R^{a1})ᵣ₁NR^{a}(CR^{a}R^{a1})ᵣ-Q, —(CR^{a}R^{a1})ᵣ₁C(O)(CR^{a}R^{a1})ᵣ-Q, —(CR^{a}R^{a1})ᵣ₁C(O)O(CR^{a}R^{a1})ᵣ-Q, —(CR^{a}R^{a1})ᵣC(O)NR^{a}R^{a1}, —(CR^{a}R^{a1})ᵣ₁C(O)NR^{a}(CR^{a}R^{a1})ᵣ-Q, —(CR^{a}R^{a1})ᵣ₁S(O)ₚ(CR^{a}R^{a1})ᵣ-Q, and —(CR^{a}R^{a1})ᵣ₁SO₂NR^{a}(CR^{a}R^{a1})ᵣ-Q;

R¹³, at each occurrence, is independently selected from H, C₁₋₆ alkylene-Q, C₂₋₆ alkenylene-Q, C₂₋₆ alkynylene-Q, —(CH₂)ᵣ₁O(CH₂)ᵣ-Q, —(CH₂)ᵣ₁NR^{a}(CH₂)ᵣ-Q, —(CH₂)ᵣ₁C(O)(CH₂)ᵣ-Q, —(CH₂)ᵣ₁C(O)NR^{a}(CH₂)ᵣ-Q, —(CH₂)ᵣ₁NR^{a}C(O)(CH₂)ᵣ-Q, —(CH₂)ᵣ₁OC(O)NR^{a}(CH₂)ᵣ-Q, —(CH₂)ᵣ₁NR^{a}C(O)O(CH₂)ᵣ-Q, —(CH₂)ᵣ₁NR^{a}C(O)NR^{a}(CH₂)ᵣ-Q, —(CH₂)ᵣ₁S(O)ₚ(CH₂)ᵣ-Q, —(CH₂)ᵣ₁SO₂NR^{a}(CH₂)ᵣ-Q, —(CH₂)ᵣ₁NR^{a}SO₂(CH₂)ᵣ-Q, and —(CH₂)ᵣ₁NR^{a}SO₂NR^{a}(CH₂)ᵣ-Q;

alternatively, R¹¹ and R¹² in Formula (V) or (VI), together with the carbon atoms to which they are attached, combine to form a 3-8 membered carbocycle or heterocycle comprising: carbon atoms, 0-2 ring heteroatoms selected from O, N, NR¹⁰, and S(O)ₚ, and 0-2 double bonds, and substituted with 0-3 R^{c};

alternatively, R¹² and R¹³ in Formula (V) or (VI), together with the carbon atom to which they are attached, combine to form a 3-8 membered carbocycle or heterocycle comprising: carbon atoms and 0-2 ring heteroatoms selected from O, N, NR¹⁰, and S(O)ₚ, and 0-2 double bonds, and substituted with 0-3 R^{c};

R¹⁴ is selected from H, C₁₋₆ alkyl substituted with 0-1 R^{b}, C₂₋₆ alkenyl substituted with 0-1 R^{b}, and C₂₋₆ alkynyl substituted with 0-1 R^{b};

R¹⁵ is selected from H, C₁₋₆ alkyl substituted with 0-1 R^{b}, C₂₋₆ alkenyl substituted with 0-1 R^{b}, and C₂₋₆ alkynyl substituted with 0-1 R^{b};

alternatively, when n is 1, R¹³ and R¹⁴ in Formula (V) or (VI), together with the carbon atom to which they are attached, combine to form a 3-8 membered carbocycle or heterocycle comprising: carbon atoms and 0-2 ring heteroatoms selected from O, N, NR¹⁰, and S(O)ₚ, and 0-2 double bonds, and substituted with 0-3 R^{c};

alternatively, when n is 1, R¹⁴ and R¹⁵ in Formula (V) or (VI), together with the carbon atom to which they are attached, combine to form a 3-8 membered carbocycle or heterocycle comprising: carbon atoms, 0-2 ring heteroatoms selected from O, N, NR¹1, and S(O)ₚ, and 0-2 double bonds, and substituted with 0-3 R^{c};

R¹⁶ is selected from H, C₁₋₄ alkyl, C₂₋₄ alkenyl, and C₂₋₄ alkynyl;

R¹⁷ is selected from H, C₁₋₄ alkyl, C₂₋₄ alkenyl, and C₂₋₄ alkynyl;

R^{a}, at each occurrence, is independently selected from H, C₁₋₄ alkyl, phenyl and benzyl;

R^{a1}, at each occurrence, is independently H or C₁₋₄ alkyl;

alternatively, R^{a} and R^{a1} when attached to a nitrogen are taken together with the nitrogen to which they are attached, form a 5 or 6 membered heterocycle comprising: carbon atoms and 0-1 additional heteroatoms selected from the group consisting of N, O, and S;

R^{a2}, at each occurrence, is independently selected from C₁₋₄ alkyl, phenyl, and benzyl;

R^{b}, at each occurrence, is independently selected from C₁₋₆ alkyl, OR^{a}, Cl, F, Br, ═O, CN, —NR^{a}R^{a1}, —C(O)R^{a}, —C(O)OR^{a}, —C(O)NR^{a}R^{a1}, —S(O)₂NR^{a}R^{a1}, —S(O)ₚR^{a2}, and CF₃;

R^{c}, at each occurrence, is independently selected from C₁₋₆ alkyl, OR^{a}, Cl, F, Br, ═O, CN, —NR^{a}R^{a1}, —C(O)R^{a}, —C(O)OR^{a}, —C(O)NR^{a}R^{a1}, —S(O)₂NR^{a}R^{a1}, —S(O)ₚR^{a2}, CF₃, —(CH₂)ᵣ—C₃₋₆ carbocycle and a —(CH₂)ᵣ-5-6 membered heterocycle comprising: carbon atoms and 1-4 heteroatoms selected from the group consisting of N, O, and S;

alternatively, two R^{c} groups on the same carbon atom are taken together with the carbon atom to which they are attached to form a 5 or 6 membered carbocycle or heterocycle comprising: carbon atoms and 0-1 additional heteroatoms selected from the group consisting of N, O, and S;

R^{c1}, at each occurrence, is independently selected from C₁₋₆ alkyl, OR^{a}, Cl, F, Br, I, ═O, CN, NO₂, —NR^{a}R^{a1}, —C(O)R^{a}, —C(O)OR^{a}, —C(O)NR^{a}R^{a1}, —NR^{a}C(O)NR^{a}R^{a1}, —OC(O)NR^{a}R^{a1}, —NR^{a}C(O)OR^{a}, —S(O)₂NR^{a}R^{a1}, —NR^{a}S(O)₂R^{a2}, —NR^{a}S(O)₂NR^{a}R^{a1}, —OS(O)₂NR^{a}R^{a1}, —NR^{a}S(O)₂R^{a2}, —S(O)ₚR^{a2}, CF₃, CF₂CF₃, CH₂F, and CHF₂;

R^{d}, at each occurrence, is independently selected from C₁₋₆ alkyl, OR^{a}, Cl, F, Br, ═O, CN, —NR^{a}R^{a1}, —C(O)R^{a}, —C(O)OR^{a}, —C(O)NR^{a}R^{a1}, —S(O)₂NR^{a}R^{a1}, —S(O)ₚR^{a2}, CF₃, C₃₋₆ carbocyclic residue and a 5-6 membered heterocycle comprising: carbon atoms and 1-4 heteroatoms selected from the group consisting of N, O, and S;

R^{e}, at each occurrence, is phenyl substituted with 0-2 R^{b} or biphenyl substituted with 0-2 R^{b};

R^{f}, at each occurrence, is C₁₋₄ alkyl, C₃₋₆ cycloalkyl, C₁₋₅ alkoxy, or phenyl substituted with 0-2 R^{b};

R^{g}, at each occurrence, is independently selected from C₁₋₆ alkyl, OR^{a}, Cl, F, Br, I, ═O, CN, NO₂, —NR^{a}R^{a1}, —C(O)R^{a}, —C(O)OR^{a}, —C(O)NR^{a}R^{a1}, —NR^{a}C(O)NR^{a}R^{a1}, —OC(O)NR^{a}R^{a1}, —NR^{a}C(O)OR^{a}, —S(O)₂NR^{a}R^{a1}, —NR^{a}S(O)₂R^{a2}, —NR^{a}S(O)₂NR^{a}R^{a1}, —OS(O)₂NR^{a}R^{a1}, —NR^{a}S(O)₂R^{a2}, —S(O)ₚR^{a2}, CF₃, CF₂CF₃, C₃₋₁₀ carbocycle substituted with 0-2 R^{c1}, —(CR^{a}R^{a1})ᵣ₁—C₃₋₁₀ carbocycle substituted with 0-2 R^{c1}, a 5-14 membered heterocycle comprising carbon atoms and 1-4 heteroatoms selected from the group consisting of N, O, and S(O)ₚ and substituted with 0-2 R^{c1}, and —(CR^{a}R^{a1})ᵣ₁-5-14 membered heterocycle comprising carbon atoms and 1-4 heteroatoms selected from the group consisting of N, O, and S(O)ₚ and substituted with 0-2 R^{c1};

R^{h}, at each occurrence, is independently selected from H, C₁₋₆ alkyl, C₁₋₆ alkoxy, phenoxy, benzoxy, C₃₋₁₀ carbocycle substituted with 0-2 R^{c1}, and a 5-10 membered heterocycle consisting of carbon atoms and 1-4 heteroatoms selected from the group consisting of N, O, and S(O)ₚ, and substituted with 0-2 R^{c1};

m, at each occurrence, is selected from 0, 1, 2 and 3;

n is 0 or 1;

p, at each occurrence, is selected from 0, 1, and 2;

r, at each occurrence, is selected from 0, 1, 2, 3, and 4;

r1, at each occurrence, is selected from 0, 1, 2, 3, and 4; and

t, at each occurrence, is selected from 1, 2, 3, and 4.

Preferred TACE inhibitors useful in the present invention are compounds selected from:

[1(R)]-3-amino-N-hydroxy-α-(2-methylpropyl)-2-oxo-3-[4-(2-quinolinylmethoxy)phenyl]-1-pyrrolidineacetamide;

[1(R)]-3-amino-N-hydroxy-α-(2-methylpropyl)-2-oxo-3-[4-(4 quinolinylmethoxy)phenyl]-1-pyrrolidineacetamide;

[1(R)]-3-amino-N-hydroxy-3-[4-[(2-methoxy-4-quinolinyl)methoxy]phenyl]-α-(2-methylpropyl)-2-oxo-1-pyrrolidineacetamide;

[1(R)]-3-amino-N-hydroxy-α-(2-methylpropyl)-2-oxo-3-[4-[(2-phenyl-4-quinolinyl)methoxy]phenyl]-1-pyrrolidineacetamide;

[1(R)]-3-amino-3-[4-[(2,6-dimethyl-4-quinolinyl)methoxy]phenyl]-N-hydroxy-α-(2-methylpropyl)-2-oxo-1-pyrrolidineacetamide;

[1(R)]-3-amino-3-[4-[(2-chloro-4-quinolinyl)methoxy]phenyl]-N-hydroxy-α-(2-methylpropyl)-2-oxo-1-pyrrolidineacetamide;

[1(R)]-3-amino-N-hydroxy-α-(2-methylpropyl)-3-[4-[(2-methyl-4-quinolinyl)methoxy]phenyl]-2-oxo-1-pyrrolidineacetamide;

[1(R)]-3-amino-3-[4-[(2-chloro-4-quinolinyl)methoxy]phenyl]-N-hydroxy-α-[2-(methylsulfonyl)ethyl]-2-oxo-1-pyrrolidineacetamide;

[1(R)]-3-amino-N-hydroxy-3-[4-[(2-methyl-4-quinolinyl)methoxy]phenyl]-α-[2-(methylsulfonyl)ethyl]-2-oxo-1-pyrrolidineacetamide;

[1(R)]-3-amino-N-hydroxy-3-[4-[(2-methoxy-4-quinolinyl)methoxy]phenyl]-x-[2-(methylsulfonyl)ethyl]-2-oxo-1-pyrrolidineacetamide;

[1(R)]-N-hydroxy-3-(methylamino)-α-(2-methylpropyl)-3-[4-[(2-methyl-4-quinolinyl)methoxy]phenyl]-2-oxo-1-pyrrolidineacetamide;

[1(R)]-α-[3-amino-2-oxo-3-[4-(4-quinolinylmethoxy)phenyl]-1-pyrrolidinyl]-N-hydroxy-4-piperidineacetamide;

[1(R)]-3-amino-N-hydroxy-x-(1-methylethyl)-2-oxo-3-[4-(4-quinolinylmethoxy)phenyl]-1-pyrrolidineacetamide;

[1(R)]-3-amino-α-cyclohexyl-N-hydroxy-2-oxo-3-[4-(4-quinolinylmethoxy)phenyl]-1-pyrrolidineacetaniide;

[1(R)]-3-amino-x-(1,1-dimethylethyl)-N-hydroxy-2-oxo-3-[4-(4-quinolinylmethoxy)phenyl]-1-pyrrolidineacetaniide;

[1(R)]-3-amino-α-(1,1-dimethylethyl)-N-hydroxy-2-oxo-3-[4-[(2-methyl-4-quinolinyl)methoxy]phenyl]-1-pyrrolidineacetainide;

[1(R)]-3-amino-N-hydroxy-α-(1-methylethyl)-2-oxo-3-[4-[(2-methyl-4-quinolinyl)methoxy]phenyl]-1-pyrrolidineacetamide;

[1(R)]-3-amino-N-hydroxy-α-(1-methylethyl)-2-oxo-3-[4-[(2,6-dimethyl-4-quinolinyl)methoxy]phenyl]-1-pyrrolidineacetamide;

(3S,4S)-N-hydroxy-1-isopropyl-4-({4-[(2-methyl-4-quinolinyl)methoxy]benzoyl}amino)-3-pyrrolidinecarboxamide;

(3S,4S)-N-hydroxy-4-({4-[(2-methyl-4-quinolinyl)methoxy]benzoyl}amino)-1-(2-propynyl)-3-pyrrolidinecarboxamide;

(3S,4S)-1-(2-butynyl)-N-hydroxy-4-({4-[(2-methyl-4-quinolinyl)methoxy]benzoyl}amino)-3-pyrrolidinecarboxamide;

(3R,4S)-N-hydroxy-4-({4-[(2-methyl-4-quinolinyl)methoxy]benzoyl}amino)tetrahydro-3-furancarboxamide;

(3S,4S)-4-{[4-(2-butynyloxy)benzoyl]amino}-N-hydroxy-1-isopropyl-3-pyrrolidinecarboxamide;

(1S,2R)-N-hydroxy-2-[[[4-[(2-methyl-4-quinolinyl)methoxy]phenyl]carbonyl]amino]-1-cyclopentanecarboxamide;

(3S,4R)-N-hydroxy-1-methyl-4-[[[4-[(2-methyl-4-quinolinyl)methoxy]phenyl]carbonyl]amino]-3-piperidinecarboxamide;

(3S,4S)-N-hydroxy-1-(1-methylethyl)-3-[[[4-[(2-methyl-4-quinolinyl)methoxy]phenyl]carbonyl]amino]-4-piperidinecarboxamiide;

(3R,4R)-N-hydroxy-4-({4-[(2-methyl-4-quinolinyl)methoxy]benzoyl}amino)tetrahydro-2H-pyran-3-carboxamide;

(3S,4S)-1-tert-butyl-N-hydroxy-3-({4-[(2-methyl-4-quinolinyl)methoxy]benzoyl}amino)-4-piperidinecarboxamide;

(3S,4S)-3-({4-[(2,5-dimethylbenzyl)oxy]benzoyl}amino)-N-hydroxy-4-piperidinecarboxamide;

N-{4-[2-(hydroxyamino)-2-oxoethyl]-4-piperidinyl}-4-[(2-methyl-4-quinolinyl)methoxy]benzamide;

N-[3-[2-(hydroxyan-ino)-2-oxoethyl]-1-(4-pyridinylmethyl)-3-piperidinyl]-4-[(2-methyl-4-quinolinyl)methoxy]benzamide;

N-{4α-[2-(hydroxyamino)-2-oxoethyl]-2β,6β-dimethyl-4-piperidinyl}-4-[(2-methyl-4-quinolinyl)methoxy]benzamide;

N-{4-[2-(hydroxyamino)-2-oxoethyl]hexahydro-1H-azepin-4-yl}-4-[(2-methyl-4-quinolinyl)methoxy]benzamide;

N-{4-[2-(hydroxyamino)-2-oxoethyl]tetrahydro-2H-pyran-4-yl}-4-[(2-methyl-4-quinolinyl)methoxy]benzamide;

N-{(3R)-3-[2-(hydroxyamino)-2-oxoethyl]tetrahydro-2H-pyran-3-yl}-4-[(2-methyl-4-quinolinyl)methoxy]benzamide;

N-{(3S)-3-[2-(hydroxyamino)-2-oxoethyl]tetrahydro-2H-pyran-3-yl}-4-[(2-methyl-4-quinolinyl)methoxy]benzamide;

N-{4-[2-(hydroxyamino)-2-oxoethyl]tetrahydro-2H-thiopyran-4-yl}-4-[(2-methyl-4-quinolinyl)methoxy]benzamide;

N-{4-[2-(hydroxyamino)-2-oxoethyl]-1,1-dioxidotetrahydro-2H-thiopyran-4-yl}-4-[(2-methyl-4-quinolinyl)methoxy]benzamide;

N-{3-[2-(hydroxyamino)-2-oxoethyl]tetrahydro-3-furanyl}-4-[(2-methyl-4-quinolinyl)methoxy]benzamide;

(5R,7S,8R)-N-hydroxy-8-({4-[(2-methyl-4-quinolinyl)methoxy]benzoyl}amino)-1-oxaspiro[4.4]nonane-7-carboxamide;

(5S,7S,8R)-N-hydroxy-8-({4-[(2-methyl-4-quinolinyl)methoxy]benzoyl}amino)-1-oxaspiro[4.4]nonane-7-carboxamide;

(5R,7S,8R)-8-{[4-(2-butynyloxy)benzoyl]amino}-N-hydroxy-1-oxaspiro[4.4]nonane-7-carboxamide;

(5R,7S,8R)-N-hydroxy-8-({4-[(2-isopropyl-1H-benzimidazol-1-yl)methyl]benzoyl}amino)-1-oxaspiro[4.4]nonane-7-carboxamide;

(5R,7S,8R)-N-hydroxy-8-[(4-{[2-(trifluoromethyl)-1H-benzimidazol-1-yl]methyl}benzoyl)amino]-1-oxaspiro[4.4]nonane-7-carboxamide;

(5R,7S,8R)-8-[(4-{[2-(11,1-difluoroethyl)-1H-benzimidazol-1-yl]methyl}benzoyl)amino]-N-hydroxy-1-oxaspiro[4.4]nonane-7-carboxamide;

(5R,7S,8R)-8-({4-[(3,5-dimethyl-1H-pyrazol-4-yl)methyl]benzoyl}amino)-N-hydroxy-1-oxaspiro[4.4]nonane-7-carboxamide;

(5R,7S,8R)-N-hydroxy-8-({4-[(2-methyl-4-quinolinyl)methyl]benzoyl}amino)-1-oxaspiro[4.4]nonane-7-carboxamide;

(5R,7S,8R)-N-hydroxy-8-[(4-{[2-(trifluoromethyl)-4-quinolinyl]methyl}benzoyl)amino]-1-oxaspiro[4.4]nonane-7-carboxamide; and

(5R,7S,8R)-N-hydroxy-8-({4-[(2-isopropyl-4-quinolinyl)methyl]benzoyl}amino)-1-oxaspiro[4.4]nonane-7-carboxamide;

or a pharmaceutically acceptable salt, hydrate, or prodrug thereof.

More preferred TACE inhibitors useful in the present invention are compounds selected from:

[1(R)]-3-amino-N-hydroxy-3-[4-[(2-methoxy-4-quinolinyl)methoxy]phenyl]-α-(2-methylpropyl)-2-oxo-1-pyrrolidineacetamide;

[1(R)]-3-amino-N-hydroxy-α-(2-methylpropyl)-2-oxo-3-[4-[(2-phenyl-4-quinolinyl)methoxy]phenyl]-1-pyrrolidineacetamide;

[1(R)]-3-amino-N-hydroxy-α-(2-methylpropyl)-3-[4-[(2-methyl-4-quinolinyl)methoxy]phenyl]-2-oxo-1-pyrrolidineacetamide;

[1(R)]-3-amino-3-[4-[(2,6-dimethyl-4-quinolinyl)methoxy]phenyl]-N-hydroxy-α-(2-methylpropyl)-2-oxo-1-pyrrolidineacetamide; and

[1(R)]-3-amino-3-[4-[(2-chloro-4-quinolinyl)methoxy]phenyl]-N-hydroxy-α-(2-methylpropyl)-2-oxo-1-pyrrolidineacetamide;

or a pharmaceutically acceptable salt, hydrate or prodrug thereof.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Geometric isomers of double bonds such as olefins and C═N double bonds can also be present in the compounds described herein, and all such stable isomers are contemplated in the present invention. C is and trans geometric isomers of the compounds of the present invention are described and may be isolated as a mixture of isomers or as separated isomeric forms. All chiral, diastereomeric, and racemic forms and all geometric isomeric forms of a structure are intended, unless the specific stereochemistry or isomeric form is specifically indicated. All processes used to prepare compounds of the present invention and intermediates made therein are considered to be part of the present invention.

The following are definitions of the terms as used throughout this specification and claims. The initial definition provided for a group or term herein applies to that group or term throughout the present specification, individually or as part of another group, unless otherwise indicated.

As used herein, “alkyl” or “alkylene” is intended to include both branched and straight-chain saturated aliphatic hydrocarbon groups having the specified number of carbon atoms. C₁₋₁₀ alkyl (or alkylene), is intended to include C₁, C₂, C₃, C₄, C₅, C₆, C₇, C₈, C₉, and C₁₀ alkyl groups. Examples of alkyl include, but are not limited to, methyl, ethyl, n-propyl, i-propyl, n-butyl, s-butyl, t-butyl, n-pentyl, and s-pentyl. “Haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, and pentachloroethyl. “Alkoxy” represents an alkyl group as defined above with the indicated number of carbon atoms attached through an oxygen bridge. C₁₋₁₀ alkoxy, is intended to include C₁, C₂, C₃, C₄, C₅, C₆, C₇, C₈, C₉, and C₁₀ alkoxy groups. Examples of alkoxy include, but are not limited to, methoxy, ethoxy, n-propoxy, i-propoxy, n-butoxy, s-butoxy, t-butoxy, n-pentoxy, and s-pentoxy. “Cycloalkyl” is intended to include saturated ring groups, such as cyclopropyl, cyclobutyl, or cyclopentyl. C₃₋₇ cycloalkyl, is intended to include C₃, C₄, C₅, C₆, and C₇ cycloalkyl groups. “Alkenyl” or “alkenylene” is intended to include hydrocarbon chains of either a straight or branched configuration and one or more unsaturated carbon-carbon bonds which may occur in any stable point along the chain, such as ethenyl and propenyl. C₂₋₁₀ alkenyl (or alkenylene), is intended to include C₂, C₃, C₄, C₅, C₆, C₇, C₈, C₉, and C₁₀ alkenyl groups. “Alkynyl” or “alkynylene” is intended to include hydrocarbon chains of either a straight or branched configuration and one or more triple carbon-carbon bonds which may occur in any stable point along the chain, such as ethynyl and propynyl. C₂₋₁₀ alkynyl (or alkynylene), is intended to include C₂, C₃, C₄, C₅, C₆, C₇, C₈, C₉, and C₁₀ alkynyl groups.

“Halo” or “halogen” as used herein refers to fluoro, chloro, bromo, and iodo; and “counterion” is used to represent a small, negatively charged species such as chloride, bromide, hydroxide, acetate, and sulfate.

As used herein, “carbocycle” or “carbocyclic residue” is intended to mean any stable 3, 4, 5, 6, or 7-membered monocyclic or bicyclic or 7, 8, 9, 10, 11, 12, or 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0)bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl.

As used herein, the term “heterocycle” or “heterocyclic group” is intended to mean a stable 5, 6, or 7-membered monocyclic or bicyclic or 7, 8, 9, or 10-membered bicyclic heterocyclic ring which is saturated, partially unsaturated or unsaturated (aromatic), and which consists of carbon atoms and 1, 2, 3, or 4 heteroatoms independently selected from the group consisting of N, O and S and including any bicyclic group in which any of the above-defined heterocyclic rings is fused to a benzene ring. The nitrogen and sulfur heteroatoms may optionally be oxidized. The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. As used herein, the term “aromatic heterocyclic group” or “heteroaryl” is intended to mean a stable 5, 6, or 7-membered monocyclic or bicyclic or 7, 8, 9, or 10-membered bicyclic heterocyclic aromatic ring which consists of carbon atoms and 1, 2, 3, or 4 heterotams independently selected from the group consisting of N, O and S. It is to be noted that total number of S and O atoms in the aromatic heterocycle is not more than 1.

Examples of heterocycles include, but are not limited to, acridinyl, azocinyl, benzimidazolyl, benzofuranyl, benzothiophenyl, benzoxazolyl, benzthiazolyl, benztriazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl, 1,1-dioxido-2,3-dihydro-4H-1,4-benzothiazin-4-yl, 1,1-dioxido-3,4-dihydro-2H-1-benzothiopyran-4-yl, 3,4-dihydro-2H-chromen-4-yl, imidazo[1,2-a]pyridinyl, imidazo[1,5-a]pyridinyl, and pyrazolo[1,5-a]pyridinyl. Also included are fused ring and spiro compounds containing, for example, the above heterocycles.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

“Therapeutically effective amount” is intended to include an amount of a compound of the present invention or an amount of the combination of compounds claimed effective to inhibit a desired metalloprotease in a mammal. The combination of compounds is preferably a synergistic combination. Synergy, as described for example by Chou and Talalay, Adv. Enzyme Regul. 22:27-55 (1984), occurs when the effect (in this case, inhibition of the desired target) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased anti-inflammatory effect, or some other beneficial effect of the combination compared with the individual components.

I claim:

1. A medical device for implanting into a mammalian body wherein the medical device has a coating material comprising an amount of a TACE inhibitor effective for reducing restinosis, or a pharmaceutically-acceptable salt, hydrate, or prodrug thereof.

2. The medical device of claim 1, wherein the medical device is a stent.

3. The stent according to claim 2, in which the TACE inhibitor is a compound having Formula (I), (II), (III), (IV), (V) or (VI): 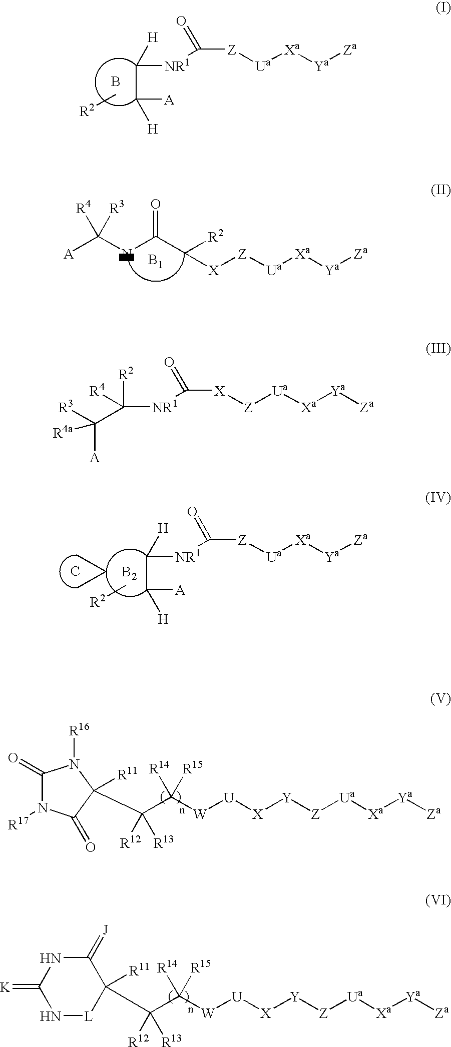
or a stereoisomer or a pharmaceutically acceptable salt, hydrate, or prodrug thereof, wherein;
A, at each occurrence, is independently selected from —COR⁵, —CO₂H, —CH₂CO₂H, —CONHOH, —CONHOR⁵, —CONHOR⁶, —N(OH)COR⁵, —SH, and —CH₂SH;
ring B is a 4-7 membered non-aromatic ring comprising: carbon atoms, 0-1 carbonyl group, 0-1 double bond, and 0-2 ring heteroatoms selected from O, N, NR¹⁰ and substituted with 0-3 R^{c}; provided that ring B contains other than a O—O or N—O bond;
ring B₁ is a 4-7 membered cyclic amide comprising: carbon atoms, 0-2 additional heteroatoms selected from O, NR¹⁰, and S(O)ₚ, 0-1 additional carbonyl group, and 0-1 double bond;
ring B₂ is a 4-7 membered non-aromatic carbocycle or heterocycle comprising: carbon atoms, 0-1 carbonyl group, 0-1 double bond, and 0-2 ring heteroatoms selected from O, N, and NR¹⁰, provided that ring B contains other than a O—O bond;
ring C forms a spiro ring on Ring B₂ and is a 4-10 membered carbocycle substituted with 0-3 R^{g} or a 4-10 membered heterocycle comprising: carbon atoms, 0-3 carbonyl groups, 0-4 double bonds, and 0-4 ring heteroatoms selected from O, N, NR¹⁰, and S(O)ₚ and substituted with 0-3 R^{g}, provided that ring C contains other than a S—S, O—O, or S—O bond;
J is O or S;
K is O or S;
L is C(═O), C(═S) or CH₂;
U, at each occurrence, is absent or is independently selected from O, NR^{a}, C(O), C(O)O, C(O)NR^{a}, NR^{a}C(O), S(O)ₚ, and S(O)ₚNR^{a};
X, at each occurrence, is absent or is independently C₁₋₄ alkylene, C₂₋₄ alkenylene, or C₂₋₄ alkynylene;
Y, at each occurrence, is absent or is independently O, NR^{a}, S(O)ₚ, or C(O);
W is (CR^{a}R^{a1})ₘ, C₂₋₃ alkenylene, or C₂₋₃ alkynylene;
Z, at each occurrence, is independently selected from a C₃₋₆ carbocycle substituted with 0-4 R^{b} and a 5-6 membered heterocycle comprising: carbon atoms, 1-4 heteroatoms selected from the group consisting of N, O, and S(O)ₚ, and substituted with 0-5 R^{b};
U^{a}, at each occurrence, is absent or is independently selected from: O, NR^{a}, C(O), C(O)O, C(O)NR^{a}, NR^{a}C(O), S(O)ₚ, and S(O)ₚNR^{a};
X^{a}, at each occurrence, is absent or is independently C₁₋₄ alkylene, C₂₋₄ alkenylene, or C₂₋₄ alkynylene;
Y^{a}, at each occurrence, is absent or is independently O or NR^{a};
Z^{a}, at each occurrence, is independently selected from H, a C₃— ₀ carbocycle substituted with 0-5 R^{c} and a 5-10 membered heterocycle comprising: carbon atoms, 1-4 heteroatoms selected from the group comprising N, O, and S(O)ₚ, and substituted with 0-5 R^{c};
provided that Z, U^{a}, Y^{a}, and Z^{a} do not combine to form a N—N, N—O, O—N, O—O, S(O)ₚ-0, O—S(O)ₚ or S(O)ₚ—S(O)ₚ group;
R¹, at each occurrence, is independently selected from H, C₁₋₄ alkyl, phenyl and benzyl;
R², at each occurrence, is independently selected from Q, C₁₋₆ alkylene-Q, C₂₋₆ alkenylene-Q, C₂₋₆ alkynylene-Q, —(CR^{a}R^{a1})ᵣ₁O(CR^{a}R^{a1})ᵣ-Q, —(CR^{a}R^{a1})ᵣ₁NR^{a}(CR^{a}R^{a1})ᵣ-Q, —(CR^{a}R^{a1})ᵣ₁C(O)(CR^{a}R^{a1})ᵣ-Q, —(CR^{a}R^{a1})ᵣ₁C(O)O(CR^{a}R^{a1})ᵣ-Q, —(CR^{a}R^{a1})ᵣC(O)NR^{a}R^{a1}, —(CR^{a}R^{a1})ᵣ₁C(O)NR^{a}(CR^{a}R^{a1})ᵣ-Q, —(CR^{a}R^{a1})ᵣ₁S(O)ₚ(CR^{a}R^{a1})ᵣ-Q, and —(CR^{a}R^{a1})ᵣ₁SO₂NR^{a}(CR^{a}R^{a1})ᵣ-Q;
R³, at each occurrence, is independently selected from H, C₁₋₆ alkylene-Q, C₂₋₆ alkenylene-Q, C₂₋₆ alkynylene-Q, —(CH₂)ᵣ₁O(CH₂)ᵣ-Q, —(CH₂)ᵣ₁NR^{a}(CH₂)ᵣ-Q, —(CH₂)ᵣ₁C(O)(CH₂)ᵣ-Q, —(CH₂)ᵣ₁C(O)NR^{a}(CH₂)ᵣ-Q, —(CH₂)ᵣ] NR^{a}C(O)(CH₂)ᵣ-Q, —(CH₂)ᵣ₁OC(O)NR^{a}(CH₂)ᵣ-Q, —(CH₂)ᵣ₁NR^{a}C(O)O(CH₂)ᵣ-Q, —(CH₂)ᵣ₁NR^{a}C(O)NR^{a}(CH₂)ᵣ-Q, —(CH₂)ᵣ₁S(O)ₚ(CH₂)ᵣ-Q, —(CH₂)ᵣ₁SO₂NR^{a}(CH₂)ᵣ-Q, —(CH₂)ᵣ₁NR^{a}SO₂(CH₂)ᵣ-Q, and —(CH₂)ᵣ₁NR^{a}SO₂NR^{a}(CH₂)ᵣ-Q;
Q, at each occurrence, is independently selected from H, a C₃₋₆ carbocycle substituted with 0-5 R^{d} and a 5-10 membered heterocycle comprising: carbon atoms and 1-4 heteroatoms selected from the group consisting of N, O, and S(O)ₚ, and substituted with 0-5 R^{d};
R⁴, at each occurrence, is independently H or C₁₋₆ alkyl;
R^{4a} is H or C₁₋₆ alkyl;
alternatively, R³ and R⁴ in Formula (II), together with the carbon atom to which they are attached, combine to form a 3-6 membered carbocycle or heterocycle comprising: carbon atoms and 0-2 ring heteroatoms selected from O, N, NR¹⁰, and S(O)ₚ, and substituted with 0-1 R^{c};
alternatively, R¹ and R² in Formula (III), together with the carbon and nitrogen atoms to which they are attached, combine to form a 3-10 membered heterocycle comprising: carbon atoms and, in addition to the nitrogen atom to which R¹ is attached, 0-1 ring heteroatoms selected from O, N, NR¹⁰, and S(O)ₚ, and substituted with 0-1 R^{c};
alternatively, R¹ and R³ in Formula (III), together with the carbon and nitrogen atoms to which they are attached, combine to form a 4-6 membered heterocycle comprising: carbon atoms and, in addition to the nitrogen atom to which R¹ is attached, 0-1 ring heteroatoms selected from O, N, and NR¹⁰, and substituted with 0-1 R^{c};
alternatively, R² and R⁴ in Formula (III), together with the carbon atom to which they are attached, combine to form a 3-10 membered carbocycle or heterocycle comprising: carbon atoms and 0-2 ring heteroatoms selected from O, N, NR¹⁰, and S(O)ₚ, and substituted with 0-3 R^{c};
alternatively, R³ and R^{4a} in Formula (III), together with the carbon atom to which they are attached, combine to form a 3-6 membered carbocycle or heterocycle comprising: carbon atoms and 0-2 ring heteroatoms selected from O, N, NR¹⁰, and S(O)ₚ and substituted with 0-1 R^{c};
R⁵, at each occurrence, is independently selected from C₁₋₆ alkyl substituted with 0-2 R^{b}, and C₁₋₄ alkyl substituted with 0-2 R^{e};
R⁶, at each occurrence, is independently selected from phenyl, naphthyl, C₁₋₁₀ alkyl-phenyl-C₁₋₆ alkyl-, C₃₋₁₁ cycloalkyl,
C₁₋₆ alkylcarbonyloxy-C₁₋₃ alkyl-, C₁₋₆ alkoxycarbonyloxy-C₁₋₃ alkyl-, C₂₋₁₀ alkoxycarbonyl, C₃₋₆ cycloalkylcarbonyloxy-C₁₋₃ alkyl-, C₃₋₆ cycloalkoxycarbonyloxy-C₁₋₃ alkyl-, C₃₋₆ cycloalkoxycarbonyl, phenoxycarbonyl, phenyloxycarbonyloxy-C₁₋₃ alkyl-, phenylcarbonyloxy-C₁₋₃ alkyl-, C₁₋₆ alkoxy-C₁₋₆ alkylcarbonyloxy-C₁₋₃ alkyl-, [5-(C₁-C₅ alkyl)-1,3-dioxa-cyclopenten-2-one-yl]methyl, [5-(R^{a})-1,3-dioxa-cyclopenten-2-one-yl]methyl, (5-aryl-1,3-dioxa-cyclopenten-2-one-yl)methyl, —C₁₋₁₀ alkyl-NR⁷R^{7a}, —CH(R⁸)OC(═O)R⁹, and —CH(R⁸)OC(═O)OR⁹;
R⁷, at each occurrence, is independently H, C₁₋₆ alkyl, C₂₋₆ alkenyl, C₃₋₆ cycloalkyl-C₁₋₃ alkyl-, or phenyl-C₁₋₆ alkyl-;
R^{7a}, at each occurrence, is independently H, C₁₋₆ alkyl, C₂₋₆ alkenyl, C₃₋₆ cycloalkyl-C₁₋₃ alkyl-, or phenyl-C₁₋₆ alkyl-;
R⁸, at each occurrence, is independently H or C₁₋₄ linear alkyl;
R⁹, at each occurrence, is independently selected from H, C₁₋₆ alkyl substituted with 1-2 R^{f}, C₃₋₆ cycloalkyl substituted with 1-2 R^{f}, and phenyl substituted with 0-2 R^{b};
R¹⁰, at each occurrence, is independently selected from H, —(CR^{a}R^{a1})ₜNR^{a}R^{a1}, —(CR^{a}R^{a1})ᵣ₁C(O)NR^{a}OH, —(CR^{a}R^{a1})ᵣ₁C(O)(CR^{a}R^{a1})ᵣR^{h}, —(CR^{a}R^{a1})ᵣC(O)OR^{a1}, —(CR^{a}R^{a1})ᵣC(S)OR^{a1}, —(CR^{a}R^{a1})ᵣC(O)NR^{a}R^{a1}, —(CR^{a}R^{a1})ₜNR^{a}C(O)R^{a1}, —(CR^{a}R^{a1})ᵣC(S)NR^{a}R^{a1}, —(CR^{a}R^{a1})ₜOC(O)NR^{a}R^{a1}, —(CR^{a}R^{a1})ₜNR^{a}C(O)OR^{a1}, —(CR^{a}R^{a1})ₜNR^{a}C(O)NR^{a}R^{a1}, —(CR^{a}R^{a1})ᵣS(O)ₚR^{a2}, —(CR^{a}R^{a1})ᵣSO₂NR^{a}R^{a1}, —(CR^{a}R^{a1})ₜNR^{a}SO₂R^{a2}, —(CR^{a}R^{a1})ₜNR^{a}SO₂NR^{a}R^{a1}, C₁₋₆ alkyl substituted with 0-2 R^{c1}, C₂₋₆ alkenyl substituted with 0-2 R^{e}, C₂₋₆ alkynyl substituted with 0-2 R^{c1}, —(CR^{a}R^{a1})ᵣ—C₃₋₁₀ carbocycle substituted with 0-2 R^{c1}, and —(CR^{a}R^{a1})ᵣ-5-10 membered heterocycle consisting of carbon atoms and 1-4 heteroatoms selected from the group consisting of N, O, and S(O)ₚ, and substituted with 0-2 R^{c1};
R¹¹ is independently selected from Q, C₁₋₆ alkylene-Q, C₂₋₆ alkenylene-Q, C₂₋₆ alkynylene-Q, —(CR^{a}R^{a1})ᵣ₁O(CR^{a}R^{a1})ᵣ-Q, —(CR^{a}R^{a1})ᵣ₁NR^{a}(CR^{a}R^{a1})ᵣ-Q, —(CR^{a}R^{a1})ᵣ₁C(O)(CR^{a}R^{a1})ᵣ-Q, —(CR^{a}R^{a1})ᵣ₁C(O)O(CR^{a}R^{a1})ᵣ-Q, —(CR^{a}R^{a1})ᵣC(O)NR^{a}R^{a1}, —(CR^{a}R^{a1})ᵣ₁C(O)NR^{a}(CR^{a}R^{a1})ᵣ-Q, —(CR^{a}R^{a1})ᵣ₁S(O)ₚ(CR^{a}R^{a1})ᵣ-Q, and —(CR^{a}R^{a1})ᵣ₁SO₂NR^{a}(CR^{a}R^{a1})ᵣ-Q;
R¹², at each occurrence, is independently selected from Q, C₁₋₆ alkylene-Q, C₂₋₆ alkenylene-Q, C₂₋₆ alkynylene-Q, —(CR^{a}R^{a1})ᵣ₁O(CR^{a}R^{a1})ᵣ-Q, —(CR^{a}R^{a1})ᵣ₁NR^{a}(CR^{a}R^{a1})ᵣ-Q, —(CR^{a}R^{a1})ᵣ₁C(O)(CR^{a}R^{a1})ᵣ-Q, —(CR^{a}R^{a1})ᵣ₁C(O)O(CR^{a}R^{a1})ᵣ-Q, —(CR^{a}R^{a1})ᵣC(O)NR^{a}R^{a1}, —(CR^{a}R^{a1})ᵣ₁C(O)NR^{a}(CR^{a}R^{a1})ᵣ-Q, —(CR^{a}R^{a1})ᵣ₁S(O)ₚ(CR^{a}R^{a1})ᵣ-Q, and —(CR^{a}R^{a1})ᵣ₁SO₂NR^{a}(CR^{a}R^{a1})ᵣ-Q;
R¹³, at each occurrence, is independently selected from H, C₁₋₆ alkylene-Q, C₂₋₆ alkenylene-Q, C₂₋₆ alkynylene-Q, —(CH₂)ᵣ₁O(CH₂)ᵣ-Q, —(CH₂)ᵣ₁NR^{a}(CH₂)ᵣ-Q, —(CH₂)ᵣ₁C(O)(CH₂)ᵣ-Q, —(CH₂)ᵣ₁C(O)NR^{a}(CH₂)ᵣ-Q, —(CH₂)ᵣ₁NR^{a}C(O)(CH₂)ᵣ-Q, —(CH₂)ᵣ₁OC(O)NR^{a}(CH₂)ᵣ-Q, —(CH₂)ᵣ₁NR^{a}C(O)O(CH₂)ᵣ-Q, —(CH₂)ᵣ₁NR^{a}C(O)NR^{a}(CH₂)ᵣ-Q, —(CH₂)ᵣ₁S(O)ₚ(CH₂)ᵣ-Q, —(CH₂)ᵣ₁SO₂NR^{a}(CH₂)ᵣ-Q, —(CH₂)ᵣ₁NR^{a}SO₂(CH₂)ᵣ-Q, and —(CH₂)ᵣ₁NR^{a}SO₂NR^{a}(CH₂)ᵣ-Q;
alternatively, R¹¹ and R¹² in Formula (V) or (VI), together with the carbon atoms to which they are attached, combine to form a 3-8 membered carbocycle or heterocycle comprising: carbon atoms, 0-2 ring heteroatoms selected from O, N, NR¹⁰, and S(O)ₚ, and 0-2 double bonds, and substituted with 0-3 R^{c};
alternatively, R¹² and R¹³ in Formula (V) or (VI), together with the carbon atom to which they are attached, combine to form a 3-8 membered carbocycle or heterocycle comprising: carbon atoms and 0-2 ring heteroatoms selected from O, N, NR¹⁰, and S(O)ₚ, and 0-2 double bonds, and substituted with 0-3 R^{c};
R¹⁴ is selected from H, C₁₋₆ alkyl substituted with 0-1 R^{b}, C₂₋₆ alkenyl substituted with 0-1 R^{b}, and C₂₋₆ alkynyl substituted with 0-1 R^{b};
R¹⁵ is selected from H, C₁₋₆ alkyl substituted with 0-1 R^{b}, C₂₋₆ alkenyl substituted with 0-1 R^{b}, and C₂₋₆ alkynyl substituted with 0-1 R^{b};
alternatively, when n is 1, R¹³ and R¹⁴ in Formula (V) or (VI), together with the carbon atom to which they are attached, combine to form a 3-8 membered carbocycle or heterocycle comprising: carbon atoms and 0-2 ring heteroatoms selected from O, N, NR¹⁰, and S(O)ₚ, and 0-2 double bonds, and substituted with 0-3 R^{c};
alternatively, when n is 1, R¹⁴ and R¹⁵ in Formula (V) or (VI), together with the carbon atom to which they are attached, combine to form a 3-8 membered carbocycle or heterocycle comprising: carbon atoms, 0-2 ring heteroatoms selected from O, N, NR¹⁰, and S(O)ₚ, and 0-2 double bonds, and substituted with 0-3 R^{c};
R¹⁶ is selected from H, C₁₋₄ alkyl, C₂₋₄ alkenyl, and C₂₋₄ alkynyl;
R¹⁷ is selected from H, C₁₋₄ alkyl, C₂₋₄ alkenyl, and C₂₋₄ alkynyl;
R^{a}, at each occurrence, is independently selected from H, C₁₋₄ alkyl, phenyl and benzyl;
R^{a1}, at each occurrence, is independently H or C₁₋₄ alkyl;
alternatively, R^{a} and R^{a1} when attached to a nitrogen are taken together with the nitrogen to which they are attached, form a 5 or 6 membered heterocycle comprising: carbon atoms and 0-1 additional heteroatoms selected from the group consisting of N, O, and S;
R^{a2}, at each occurrence, is independently selected from C₁₋₄ alkyl, phenyl, and benzyl;
R^{b}, at each occurrence, is independently selected from C₁₋₆ alkyl, OR^{a}, Cl, F, Br, ═O, CN, —NR^{a}R^{a1}, —C(O)R^{a}, —C(O)OR^{a}, —C(O)NR^{a}R^{a1}, —S(O)₂NR^{a}R^{a1}, —S(O)ₚR^{a2}, and CF₃;
R^{c}, at each occurrence, is independently selected from C₁₋₆ alkyl, OR^{a}, Cl, F, Br, ═O, CN, —NR^{a}R^{a1}, —C(O)R^{a}, —C(O)OR^{a}, —C(O)NR^{a}R^{a1}, —S(O)₂NR^{a}R^{a1}, —S(O)ₚR^{a2}, CF₃, —(CH₂)ᵣ—C₃₋₆ carbocycle and a —(CH₂)ᵣ-5-6 membered heterocycle comprising: carbon atoms and 1-4 heteroatoms selected from the group consisting of N, O, and S;
alternatively, two R^{c} groups on the same carbon atom are taken together with the carbon atom to which they are attached to form a 5 or 6 membered carbocycle or heterocycle comprising: carbon atoms and 0-1 additional heteroatoms selected from the group consisting of N, O, and S;
R^{c1}, at each occurrence, is independently selected from C₁₋₆ alkyl, OR^{a}, Cl, F, Br, I, ═O, CN, NO₂, —NR^{a}R^{a1}, —C(O)R^{a}, —C(O)OR^{a}, —C(O)NR^{a}R^{a1}, —NR^{a}C(O)NR^{a}R^{a1}, —OC(O)NR^{a}R^{a1}, —NR^{a}C(O)OR^{a}, —S(O)₂NR^{a}R^{a1}, —NR^{a}S(O)₂R^{a2}, —NR^{a}S(O)₂NR^{a}R^{a1}, —OS(O)₂NR^{a}R^{a1}, —NR^{a}S(O)₂R^{a2}, —S(O)ₚR^{a2}, CF₃, CF₂CF₃, CH₂F, and CHF₂;
R^{d}, at each occurrence, is independently selected from C₁₋₆ alkyl, OR^{a}, Cl, F, Br, ═O, CN, —NR^{a}R^{a1}, —C(O)R^{a}, —C(O)OR^{a}, —C(O)NR^{a}R^{a1}, —S(O)₂NR^{a}R^{a1}, —S(O)ₚR^{a2}, CF₃, C₃₋₆ carbocyclic residue and a 5-6 membered heterocycle comprising: carbon atoms and 1-4 heteroatoms selected from the group consisting of N, O, and S;
R^{e}, at each occurrence, is phenyl substituted with 0-2 R^{b} or biphenyl substituted with 0-2 R^{b};
R^{f}, at each occurrence, is C₁₋₄ alkyl, C₃₋₆ cycloalkyl, C₁₋₅ alkoxy, or phenyl substituted with 0-2 R^{b};
R^{g}, at each occurrence, is independently selected from C₁₋₆ alkyl, OR^{a}, Cl, F, Br, I, ═O, CN, NO₂, —NR^{a}R^{a1}, —C(O)R^{a}, —C(O)OR^{a}, —C(O)NR^{a}R^{a1}, —NR^{a}C(O)NR^{a}R^{a1}, —OC(O)NR^{a}R^{a1}, —NR^{a}C(O)OR^{a}, —S(O)₂NR^{a}R^{a1}, —NR^{a}S(O)₂R^{a2}, —NR^{a}S(O)₂NR^{a}R^{a1}, —OS(O)₂NR^{a}R^{a1}, —NR^{a}S(O)₂R^{a2}, —S(O)ₚR^{a2}, CF₃, CF₂CF₃,
C₃₋₁₀ carbocycle substituted with 0-2 R^{c1}, —(CR^{a}R^{a1})ᵣ₁—C₃₋₁₀ carbocycle substituted with 0-2 R^{c1}, a 5-14 membered heterocycle comprising carbon atoms and 1-4 heteroatoms selected from the group consisting of N, O, and S(O)ₚ and substituted with 0-2 R^{c1}, and —(CR^{a}R^{a1})ᵣ₁-5-14 membered heterocycle comprising carbon atoms and 1-4 heteroatoms selected from the group consisting of N, O, and S(O)ₚ and substituted with 0-2 R^{c1};
R^{h}, at each occurrence, is independently selected from H, C₁₋₆ alkyl, C₁₋₆ alkoxy, phenoxy, benzoxy, C₃₋₁₀ carbocycle substituted with 0-2 R^{c1}, and a 5-10 membered heterocycle consisting of carbon atoms and 1-4 heteroatoms selected from the group consisting of N, O, and S(O)ₚ, and substituted with 0-2 R^{c1};
m, at each occurrence, is selected from 0, 1, 2 and 3;
n is 0 or 1;
p, at each occurrence, is selected from 0, 1, and 2;
r, at each occurrence, is selected from 0, 1, 2, 3, and 4;
r1, at each occurrence, is selected from 0, 1, 2, 3, and 4; and
t, at each occurrence, is selected from 1, 2, 3, and 4.

4. The stent according to claim 2, in which the TACE inhibitor is a compound selected from:
[1(R)]-3-amino-N-hydroxy-x-(2-methylpropyl)-2-oxo-3-[4-(2-quinolinylmethoxy)phenyl]-1-pyrrolidineacetamide;
[1(R)]-3-amino-N-hydroxy-α-(2-methylpropyl)-2-oxo-3-[4-(4 quinolinylmethoxy)phenyl]-1-pyrrolidineacetamide;
[1(R)]-3-amino-N-hydroxy-3-[4-[(2-methoxy-4-quinolinyl)methoxy]phenyl]-α-(2-methylpropyl)-2-oxo-1-pyrrolidineacetamide;
[1(R)]-3-amino-N-hydroxy-x-(2-methylpropyl)-2-oxo-3-[4-[(2-phenyl-4-quinolinyl)methoxy]phenyl]-1-pyrrolidineacetamide;
[1(R)]-3-amino-3-[4-[(2,6-dimethyl-4-quinolinyl)methoxy]phenyl]-N-hydroxy-α-(2-methylpropyl)-2-oxo-1-pyrrolidineacetamide;
[1(R)]-3-amino-3-[4-[(2-chloro-4-quinolinyl)methoxy]phenyl]-N-hydroxy-x-(2-methylpropyl)-2-oxo-1-pyrrolidineacetamide;
[1(R)]-3-amino-N-hydroxy-x-(2-methylpropyl)-3-[4-[(2-methyl-4-quinolinyl)methoxy]phenyl]-2-oxo-1-pyrrolidineacetamide;
[1(R)]-3-amino-3-[4-[(2-chloro-4-quinolinyl)methoxy]phenyl]-N-hydroxy-α-[2-(methylsulfonyl)ethyl]-2-oxo-1-pyrrolidineacetamide;
[1(R)]-3-amino-N-hydroxy-3-[4-[(2-methyl-4-quinolinyl)methoxy]phenyl]-α-[2-(methylsulfonyl)ethyl]-2-oxo-1-pyrrolidineacetamide;
[1(R)]-3-amino-N-hydroxy-3-[4-[(2-methoxy-4-quinolinyl)methoxy]phenyl]-α-[2-(methylsulfonyl)ethyl]-2-oxo-1-pyrrolidineacetamide;
[1(R)]-N-hydroxy-3-(methylamino)-α-(2-methylpropyl)-3-[4-[(2-methyl-4-quinolinyl)methoxy]phenyl]-2-oxo-1-pyrrolidineacetamide;
[1(R)]-α-[3-amino-2-oxo-3-[4-(4-quinolinylmethoxy)phenyl]-1-pyrrolidinyl]-N-hydroxy-4-piperidineacetamide;
[1(R)]-3-amino-N-hydroxy-(1-methylethyl)-2-oxo-3-[4-(4-quinolinylmethoxy)phenyl]-1-pyrrolidineacetamide;
[1(R)]-3-amino-α-cyclohexyl-N-hydroxy-2-oxo-3-[4-(4-quinolinylmethoxy)phenyl]-1-pyrrolidineacetamide;
[1(R)]-3-amino-x-(1,1-dimethylethyl)-N-hydroxy-2-oxo-3-[4-(4-quinolinylmethoxy)phenyl]-1-pyrrolidineacetamide;
[1(R)]-3-amino-x-(1,1-dimethylethyl)-N-hydroxy-2-oxo-3-[4-[(2-methyl-4-quinolinyl)methoxy]phenyl]-1-pyrrolidineacetamide;
[1(R)]-3-amino-N-hydroxy-(1-methylethyl)-2-oxo-3-[4-[(2-methyl-4-quinolinyl)methoxy]phenyl]-1-pyrrolidineacetamide;
[1(R)]-3-amino-N-hydroxy-x-(1-methylethyl)-2-oxo-3-[4-[(2,6-dimethyl-4-quinolinyl)methoxy]phenyl]-1-pyrrolidineacetamide;
(3S,4S)-N-hydroxy-1-isopropyl-4-({4-[(2-methyl-4-quinolinyl)methoxy]benzoyl}amino)-3-pyrrolidinecarboxamide;
(3S,4S)-N-hydroxy-4-({4-[(2-methyl-4-quinolinyl)methoxy]benzoyl}amino)-1-(2-propynyl)-3-pyrrolidinecarboxamide;
(3S,4S)-1-(2-butynyl)-N-hydroxy-4-({4-[(2-methyl-4-quinolinyl)methoxy]benzoyl}amino)-3-pyrrolidinecarboxamide;
(3R,4S)-N-hydroxy-4-({4-[(2-methyl-4-quinolinyl)methoxy]benzoyl}amino)tetrahydro-3-furancarboxamide;
(3S,4S)-4-{[4-(2-butynyloxy)benzoyl]amino}-N-hydroxy-1-isopropyl-3-pyrrolidinecarboxamide;
(1S,2R)-N-hydroxy-2-[[[4-[(2-methyl-4-quinolinyl)methoxy]phenyl]carbonyl]amino]-1-cyclopentanecarboxamide;
(3S,4R)-N-hydroxy-1-methyl-4-[[[4-[(2-methyl-4-quinolinyl)methoxy]phenyl]carbonyl]amino]-3-piperidinecarboxamide;
(3S,4S)-N-hydroxy-1-(1-methylethyl)-3-[[[4-[(2-methyl-4-quinolinyl)methoxy]phenyl]carbonyl]amino]-4-piperidinecarboxamide;
(3R,4R)-N-hydroxy-4-({4-[(2-methyl-4-quinolinyl)methoxy]benzoyl} amino)tetrahydro-2H-pyran-3-carboxamide;
(3S,4S)-1-tert-butyl-N-hydroxy-3-({4-[(2-methyl-4-quinolinyl)methoxy]benzoyl}amino)-4-piperidinecarboxamide;
(3S,4S)-3-({4-[(2,5-dimethylbenzyl)oxy]benzoyl}amino)-N-hydroxy-4-piperidinecarboxamide;
N-{4-[2-(hydroxyamino)-2-oxoethyl]-4-piperidinyl}-4-[(2-methyl-4-quinolinyl)methoxy]benzamide;
N-[3-[2-(hydroxyamino)-2-oxoethyl]-1-(4-pyridinylmethyl)-3-piperidinyl]-4-[(2-methyl-4-quinolinyl)methoxy]benzamide;
N-{4α-[2-(hydroxyamino)-2-oxoethyl]-2β,6β-dimethyl-4-piperidinyl}-4-[(2-methyl-4-quinolinyl)methoxy]benzamide;
N-{4-[2-(hydroxyamino)-2-oxoethyl]hexahydro-1H-azepin-4-yl}-4-[(2-methyl-4-quinolinyl)methoxy]benzamide;
N-{4-[2-(hydroxyamino)-2-oxoethyl]tetrahydro-2H-pyran-4-yl}-4-[(2-methyl-4-quinolinyl)methoxy]benzamide;
N-{(3R)-3-[2-(hydroxyamino)-2-oxoethyl]tetrahydro-2H-pyran-3-yl}-4-[(2-methyl-4-quinolinyl)methoxy]benzamide;
N-{(3S)-3-[2-(hydroxyamino)-2-oxoethyl]tetrahydro-2H-pyran-3-yl}-4-[(2-methyl-4-quinolinyl)methoxy]benzamide;
N-{4-[2-(hydroxyamino)-2-oxoethyl]tetrahydro-2H-thiopyran-4-yl}-4-[(2-methyl-4-quinolinyl)methoxy]benzamide;
N-{4-[2-(hydroxyamino)-2-oxoethyl]-1,1-dioxidotetrahydro-2H-thiopyran-4-yl}-4-[(2-methyl-4-quinolinyl)methoxy]benzamide;
N-{3-[2-(hydroxyamino)-2-oxoethyl]tetrahydro-3-furanyl}-4-[(2-methyl-4-quinolinyl)methoxy]benzamide;
(5R,7S,8R)-N-hydroxy-8-({4-[(2-methyl-4-quinolinyl)methoxy]benzoyl}amino)-1-oxaspiro[4.4]nonane-7-carboxamide;
(5S,7S,8R)-N-hydroxy-8-({4-[(2-methyl-4-quinolinyl)methoxy]benzoyl}amino)-1-oxaspiro[4.4]nonane-7-carboxamide;
(5R,7S,8R)-8-{[4-(2-butynyloxy)benzoyl]amino}-N-hydroxy-1-oxaspiro[4.4]nonane-7-carboxamide;
(5R,7S,8R)-N-hydroxy-8-({4-[(2-isopropyl-1H-benzimidazol-1-yl)methyl]benzoyl}amino)-1-oxaspiro[4.4]nonane-7-carboxamide;
(5R,7S,8R)-N-hydroxy-8-[(4-{[2-(trifluoromethyl)-1H-benzimidazol-1-yl]methyl}benzoyl)amino]-1-oxaspiro [4.4]nonane-7-carboxamide;
(5R,7S,8R)-8-[(4-{[2-(1,1-difluoroethyl)-1H-benzimidazol-1-yl]methyl}benzoyl)amino]-N-hydroxy-1-oxaspiro[4.4]nonane-7-carboxamide;
(5R,7S,8R)-8-({4-[(3,5-dimethyl-1H-pyrazol-4-yl)methyl]benzoyl}amino)-N-hydroxy-1-oxaspiro[4.4]nonane-7-carboxamide;
(5R,7S,8R)-N-hydroxy-8-({4-[(2-methyl-4-quinolinyl)methyl]benzoyl}amino)-1-oxaspiro[4.4]nonane-7-carboxamide;
(5R,7S,8R)-N-hydroxy-8-[(4-{[2-(trifluoromethyl)-4-quinolinyl]methyl}benzoyl)amino]-1-oxaspiro[4.4]nonane-7-carboxamide; and
(5R,7S,8R)-N-hydroxy-8-({4-[(2-isopropyl-4-quinolinyl)methyl]benzoyl}amino)-1-oxaspiro[4.4]nonane-7-carboxamide;
or a pharmaceutically acceptable salt, hydrate, or prodrug thereof.

5. The stent according to claim 4, in which the TACE inhibitor is a compound selected from:
[1(R)]-3-amino-N-hydroxy-3-[4-[(2-methoxy-4-quinolinyl)methoxy]phenyl]-α-(2-methylpropyl)-2-oxo-1-pyrrolidineacetamide;
[1(R)]-3-amino-N-hydroxy-α-(2-methylpropyl)-2-oxo-3-[4-[(2-phenyl-4-quinolinyl)methoxy]phenyl]-1-pyrrolidineacetamide;
[1(R)]-3-amino-N-hydroxy-α-(2-methylpropyl)-3-[4-[(2-methyl-4-quinolinyl)methoxy]phenyl]-2-oxo-1-pyrrolidineacetamide;
[1(R)]-3-amino-3-[4-[(2,6-dimethyl-4-quinolinyl)methoxy]phenyl]-N-hydroxy-α-(2-methylpropyl)-2-oxo-1-pyrrolidineacetamide; and
[1(R)]-3-amino-3-[4-[(2-chloro-4-quinolinyl)methoxy]phenyl]-N-hydroxy-((2-methylpropyl)-2-oxo-1-pyrrolidineacetamide;
or a pharmaceutically acceptable salt, hydrate or prodrug thereof.
